(19) United States
(12) Patent Application Publication
(10) Pub. No.: US 2003/0180775 A1
(43) Pub. Date: Sep. 25, 2003

(54) Filtered shotgun sequencing of complex eukaryotic genomes

(75) Inventors: W. Richard McCombie, Cold Spring Harbor, NY (US); Robert A. Martienssen, Cold Spring Harbor, NY (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cold Spring Harbor Laboratory, Cold Spring Harbor, NY

(21) Appl. No.: 10/371,539

(22) Filed: Feb. 20, 2003

Related U.S. Application Data
(63) Continuation of application No. 09/713,426, filed on Nov. 15, 2000.
Application No. 09/713,426 is a continuation-in-part of application No. 09/430,409, filed on Oct. 29, 1999.
(60) Provisional application No. 60/121,453, filed on Feb. 24, 1999.

(51) Int. Cl. C12Q 1/68; C12P 19/34

(52) U.S. Cl. 435/6; 435/91.2

(57) ABSTRACT

This invention provides methods by which repetitive elements can be selectively removed from genomic libraries made from complex eukaryotic genomes. In particular, the invention relates to affecting the efficiency of recovery of novel genes and regulatory sequences, by use of methylation restrictive bacteria, cell-free enzymes, and methyl-binding proteins.

### CROSS-REFERENCE TO RELATED APPLICATION

This application is a continuation-in-part of U.S. patent application, Ser. No. 09/430,409 filed Oct. 29, 1999, which is a continuation of co-pending U.S. Provisional Application, Serial No. 60/121,453, filed Feb. 24, 1999, the disclosures of which are hereby specifically incorporated by reference.

### GRANT REFERENCE

Work for this invention was funded in part by a grant from the United States Department of Agriculture, Agricultural Research Service Grant #97-35300-4564. The Government may have certain rights in this invention.

### FIELD OF THE INVENTION

This invention relates generally to the field of DNA sequencing and genomic mapping. More specifically, the invention relates to methods for rapidly identifying and localizing novel gene coding and regulatory sequences in complex eukaryotic genomes, especially genomes of plants. The invention provides methods by which highly repetitive DNA segments, segments that rarely encode expressed genes or regulatory sequences can be selectively removed from genomic libraries made from complex eukaryotic genomes.

### BACKGROUND OF THE INVENTION

The ability to analyze entire genomes is accelerating gene discovery and revolutionizing the breadth and depth of biological questions that can be addressed in model organisms, such as Saccharomyces cerevisiae, Caenorhabditis elegans, and Arabidopsis thaliana. The recent completion of the genome sequences of several microorganisms and lower eukaryotes has confirmed the view that acquisition of comprehensive genome sequences for large complex genomes, such as those found in higher eukaryotes (e.g. humans and crop plants), will have unprecedented impact and long-lasting value for basic biology, agriculture, industry, and human health.

However, the task before the genomicists is formidable. Even the smaller eukaryotic genomes are large in comparison to the prokaryotic genomes—and this is particularly true of certain agronomic plant species where ploidy is typically multiple. Arabidopsis is estimated to possess 130 Mb of genomic DNA representing 20,000 gene sequences, while rice may have as much as 400 Mb and at least 30,000 gene sequences, possibly more. Even these plants pale in view of Zea mays with an estimated 2,500 Mb of genomic DNA and an unknown number of gene sequences, and wheat with an estimated 15,000-20,000 MB of genomic sequences.

Complete analysis of an organism's genome requires extensive isolation, purification and analysis of fragments of DNA to create genomic libraries. Typically fragments as large as possible are used to minimize the number necessary to comprise the genome. The cloning systems used to generate these genomic libraries include the use of bacteriophage cosmid BAC and P1 vectors. Strains of the bacterium Escherichia coli are generally used as the host for the introduction of cloning vectors containing the DNA of interest. Most commercial strains used for cloning have been selected to preserve the integrity of the cloned DNA by eliminating certain DNA restriction systems from the bacterial genome. This is deemed especially important when cloning heterologous eukaryotic DNA into the prokaryotic cells.

Putting together the cloned genome requires ordering and linking together all of the clones comprising the genomic DNA library. Mapping strategies can be “top-down” or “bottom-up”. The “top-down” strategy depends on the separation on pulsed field gels of large DNA fragments generated using rare restriction endonucleases for physical linkage of DNA markers and construction of a long-range map. (See, e.g., Burke, et al. (1987) Science 236:806; Southern, et al. (1987) Nucleic Acids Res. 15:5925; Schwartz, et al. (1984) Cell 37:67). (See FIG. 1).

The “bottom-up” strategy depends on identifying overlapping sequences in a large number of randomly selected clones by unique restriction enzyme “fingerprinting” and their assembly into overlapping sets of clones. The linking of these clones is not done physically, but in computers and requires the analysis of thousands of individual clones to generate complete maps. Reassembled contiguous stretches of DNA are called “contigs” (See, e.g., Watson, J. D. et al (1992) Recombinant DNA, (W. H. Freeman and Company, New York), pp. 583-618, which is specifically incorporated herein by reference). Regardless of the linking strategy, the common prior art approach relied on using as large of a fragment as possible in order to minimize the numbers of “puzzle pieces” that had to be linked to obtain the genomic map.

Thus, the approach presently being taken for sequencing complex eukaryotic genomes is the same as that used for the less complex eukaryotic genomes of S. cerevisiae and C. elegans genomes, namely construction of overlapping arrays of very large insert E. coli clones (using inserts sized much larger than the average sized coding region for genes in these genomes), followed by complete sequencing of these clones one at a time. This process is labor intensive and expensive because the difficulties increase rapidly with larger genomes, requiring continual advances in mapping approaches, instrumentation and computational expertise (See, e.g., Venter, J. C., et al. (1998) Science 280:1540). For example in humans, sequence tagged sites (STSs) content mapping has proven to be an efficient method for the assembly of low resolution maps of human chromosomes Y and 21 (See Foote, et al. (1992) Science 258:60-66; Chumakov et al. (1992) Nature 358:380-387). Unfortunately, this method is limited by the lack of large numbers of suitable STS markers that can be used as reagents in large scale mapping projects designed to provide high resolution genomic maps.

Consequently, a number of strategies for preferentially sequencing genes from complex genomes have been developed. For example, cloning an unknown gene via “reverse genetics” or “positional cloning” requires identification of ever closer flanking polymorphic markers that recombine ever less frequently until candidate genes can be isolated and sequenced in mutant and wild-type populations.

Another strategy is single-pass, partial sequencing of complementary DNA (cDNA) clones to generate expressed sequence tags (ESTs; an EST is a segment of a sequence from a cDNA clone that corresponds to a messenger RNA(mRNA) (See, e.g., Adams, M. D., et al. (1991) Science 252:1651-1656; Adams, M. D., et al., (1995) Nature 377: 3174). Messenger RNA is the intermediate molecule via which the genetic information contained in DNA is transferred into proteins. Because the EST approach avoids sequencing intergenic and non-coding DNA sequences, it enables rapid identification of genes. The problem with the EST approach is that a large number of certain genes are over-represented, while environmentally or developmentally regulated genes are underrepresented, if present at all. This often results in large EST sets that they sample less than 50% of the gene complement and even then do so only with a partial coverage of each gene.

Yet another alternative approach involves sequencing all of the naturally occurring DNA sequences (i.e. genomic DNA) constituting the genome of an organism without prior mapping of large clones. Such whole genome shotgun sequencing approaches avoid the difficulty of finding every mRNA expressed in all tissues, cell types, and developmental stages. Additionally, this approach yields valuable information concerning non-coding DNA regions, including control and regulatory sequences missed by the EST approach.

Publication of the first genome from a self-replicating organism, Haemophilus influenzae, was based on such a whole-genome shotgun method (See Fleischmann, R., et al. (1995) Science, 269:496). Eight additional genomes have since been completed by this method and several others are nearing completion (See Venter, J. C., et al. (1998) Science, 280:1540-1542). In humans, it has been proposed that whole-genome shotgun sequencing would be less costly and more informative than clone-by-clone methods. (See, e.g. Weber, J. L. and E. W. Myers, (1997) Genome Research, 7:401-409).

Whole-genome shotgun sequencing essentially involves randomly breaking DNA into segments of various sizes and cloning these fragments into vectors. The clones are sequenced from both ends improving the efficiency of sequence overlapping assembly. Use of relatively long insert subclones aids in the assembly of sequences containing interspersed repetitive sequences (See, e.g. Venter, J. C., et al. (1998) Science, 280:1540-1542;,Weber, J. L. and E. W. Myers, (1997) Genome Research, 7:401-409).

A disadvantage associated with genomic shotgun sequencing approaches is the difficulty in isolating genes due to the high proportion of clones containing repetitive sequences. Repetitive sequences are often not transcribed into mRNA (i.e. “expressed”), making them of less interest in the overall goal of locating and sequencing expressed genes and the sequences that regulate them. Moreover, such repetitive sequences are dispersed throughout eukaryotic genomes making their avoidance in shotgun sequencing methods problematic. Their presence results in very low density of expressed genes in the shotgun clones, complicating genome sequencing. In one regard, this is because many of the resulting clones cannot be assembled into contigs due to the high degree of conservation between high-copy repeats. As an example, the economically important corn genome is estimated to be comprised of 50%-80% repetitive elements. (SanMiguel et al., (1996) Science 274:765-768).

As can be seen from the foregoing discussion, determining the complete sequence of complex plant and mammalian genomes to a high standard of accuracy and correspondence with the genetic map remains a considerable problem. Even the identification of a large percentage of the unique coding regions is problematic in very large genomes such as that of corn. Thus, a need exists in the art for a sequencing method that can lead to the rapid identification of genes and regulatory sequences in complex eukaryotic genomes. In particular, there is a need to combine the high throughput results obtained with genomic shotgun cloning and the specific expression mapping techniques such as ESTs.

It is an object of the present invention to provide a method of sequencing large genomes that greatly improves efficiency by removing repeat sequences from whole genomic libraries.

It is another object of the present invention to increase the number of DNA segments containing genes detected from a target genome of interest to yield all or most of the genetic information sought from the target genome, without extraneous sequence.

It is yet another object of this invention to enrich for low copy non-repeat DNA segments to be used as hybridization probes for the detection of genomic or complementary DNA sequences in arrays of single sequence clones or mixtures of sequences derived from tissue samples.

It is yet another object of this invention to create libraries of gene enriched sequences that can be compared to the genomes of other organisms to identify regions of biological importance due to the presence of shared sequence homology.

It is yet another object of this invention to create a database of nucleotide sequences (and thus corresponding predicted amino acid sequences) that is comprised of the sequence clones that have been selected in this manner.

It is yet another object of this invention to identify sequence polymorphisms in single copy DNA regions that could aid in the assembly of genetic maps or in plant breeding programs.

It is yet another object of the invention to provide genetic information which can be used in any of a number of standard assays in the art such as generation of nucleotide databases, DNA arrays or chips etc.

Other objects of the invention will become apparent from the description of the invention that which follows.

### SUMMARY OF THE INVENTION

In one regard, the present invention comprises a rapid and powerful genomic sequencing or mapping method directed toward identifying novel genes, polypeptides and regulatory sequences in complex eukaryotic genomes, especially plants. In particular, this invention relates to selectively removing repetitive elements from genomic libraries made from large complex eukaryotic genomes, especially plants, to greatly improve efficiency of sequencing.

### BRIEF DESCRIPTION OF DRAWINGS

FIG. 1 is a comparison between typical results obtained using the methods of the present invention (genetically filtered shotgun sequencing) with those results obtained typically using BAC shotgun sequencing, whole genome shotgun sequencing, and expressed sequence tag sequencing.

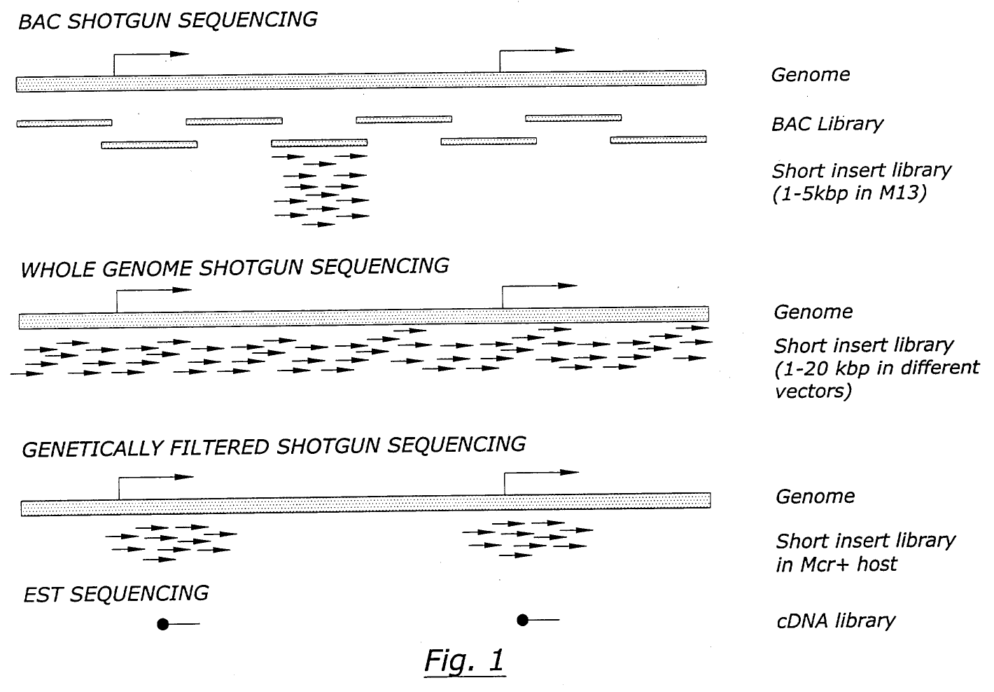

FIG. 2 (PRIOR ART) is a drawing which shows the maize genome: retro-transposable elements and other repeats are mostly confined to intergenic regions.

FIG. 3 shows dot blots of cloned sequences in the four different libraries. One 96-well filter from each library is shown [(A) JM107MA2, (B) JM101, (C) JM109, (D) JM107], hybridized with vector DNA or with maize genomic DNA radiolabeled as a probe.

FIG. 4 shows a graphical comparison of gene representation in filtered maize libraries with random rice genomic clones. (A) shows the proportions of exons and repeats in each library. (B) shows the proportion of low, medium and high copy sequences determined by hybridization.

FIG. 5 is a bar graph showing maize with/without methyl filtration, rice and Arabidopsis BAC ends technique as they each relate to annotated repeats, and unnotated repeats, minisatellite, known exons, hypothetical exons, total exons, and organellar DNA.

FIG. 6 is a three-dimensional bar graph showing the control and three test strains versus percentage of genome, versus HC, MC, LC frequencies.

FIG. 7 is a two dimensional bar graph of Zea mays only, filtered, unfiltered and two versions of partially filtered, percentages of genome, and total repeats, organellar DNA, minisatellite DNA and total exons.

FIG. 8 is a bar graph showing what portion of the total genome (in percentages) is represented by high copy, medium copy and low copy DNA for each of filtered, two versions of partially filtered, and unfiltered treatments.

FIG. 9 depicts southern hybridization gels with novel clones, where individual clones were amplified using PCR, and then used as probes on southerns, LC probes gave single copy signals while medium copy probes gave multiple signals.

### DETAILED DESCRIPTION OF THE INVENTION

The present invention is an improved method for the easy and rapid identification of novel genes and regulatory sequences in complex eukaryotic genomes. The identification method is based on the ability to exclude methylated repeat sequences from genomic libraries by the selection or engineering of an appropriate host strain. As a consequence, representative of gene-rich (i.e. low copy) sequences is greatly increased.

In one aspect the invention relies on properties which have been confirmed by the inventors to be unique to repetitive sequences to selectively exclude as many as possible from libraries. The repetitive sequences present in plant and mammalian genomes are characterized by a number of properties including high copy number, high levels of cytosine and low transcriptional activity (See, e.g., Martienssen, R. A. (1998) Trends Genet. 14:263; Kass, S. U., et al. (1997) Trends Genet. 13:335; SanMiguel, P., et al., (1996) Science 274:765; Timmermans, M. C., et al. (1996) Genetics 143:1771; Martienssen, R. A. and E. J. Richards, (1995) Curr. Opin. Genet. Dev. 5:234-242; Bennetzen, J. L., et al. (1994) Genome 37:565; White, L. F., et al. (1994) Proc. Natl. Acad. Sci. U.S.A. 91:11792; Moore, G., et al. Genomics 15:472). It had been speculated that that high copy DNA sequences often appeared to be methylated and that such sequences did not appear to be areas in which expressed genes were likely to occur. The inventors wondered if it was possible to eliminate such high copy methylated DNA from a library whether that library would be enriched for low copy DNA. The inventors postulated that one method for eliminating methylated DNA form such a library might be to “filter” such DNA through hosts capable of restricting methylated DNA.

In one embodiment the invention comprises propagation of partial genomic libraries in methylation restrictive hosts to yield fewer clones containing repetitive DNA and more clones containing expressed gene sequences. In another embodiment the invention provides libraries of polypeptides encoded thereby. One non-limiting example of a methylation restrictive host strain useful in the methods of the invention isE. coli JM107.

Bacterial strains having such genotypes are, without limitation, JM101, JM107, and JM109.

The methods of the invention will find particular usefulness in analyzing complex plant genomes. The principal example shown below deals with corn, but may be applied where the genome of interest is any cereal grain genome. Other agronomic species amenable to the methods include rice, Brassica, soybean, and wheat. And, the methods are not limited to plant genomes, but may be extended to a mammalian genome.

Also disclosed herein are methods for obtaining a hybridization probe by enriching for non repeat DNA segments. In such methods, one constructs a genomic library in a methylation restrictive host strain by inserting genomic DNA into a suitable vector, so that the inserted genomic DNA may be identified as a probe for low copy expressed gene sequences.

Also made possible by the present invention are nucleotide sequences, amino acid sequences, probes, primers, and DNA chips resulting from the application of the methods herein. Moreover, databases are now made possible comprising the nucleotide or amino acid sequences discovered by application of the methods of the invention.

“Methylation restrictive hosts”, as used herein shall include any host microorganism that is characterized by a modification-restriction phenotype such as that encoded by the mcrA, mcrBC and other methylation restriction gene products. McrA and McrBC enzymes cut methylated DNA. It is known, for instance, that McrBC sites [A/C)-mC-N(40-80)-A/C)-mC] occur every 50 bp or so in maize DNA. The mcrABC system severly restricts bacterial transformation with plant and mammalian DNA (most commercially available cloning hosts are mcrA, mcrBC in order to avoid such restriction). The mcrBC gene products specifically restrict methylated DNA, requiring two 5′Pu-mC dinucleotides separated by 40 to 80 base pairs for restriction (See Sutherland, L., et al., (1992) J. Mol. Biol. 225:327). One example of such a host isE. coli JM107.

Thus, using the methods of the present invention, methylated repetitive DNA will be underrepresented or “filtered” from libraries made in methylation restrictive hosts.

According to the invention, and to limit the probability of cloning a genome fragment that contains repetitive sequences, genetically filtered libraries are constructed by limiting insert size to that which is smaller that the average gene size for a particular genome. This would be around approximately 0.5 to about 4 kbp if the DNA is cleaved with methylation insensitive restriction enzyme and 1.6 to 4 kbp if the DNA is randomly sheared for maize. In the case of sheared libraries, removal of repetitive sequences has the added advantage of facilitating automated assembly of shotgun reads into gene-containing contigs.

In yet another preferred embodiment the information gathered in accordance with the present invention can be used in any of a number of ways standard in the art. For example it could be used to generate a database of sequences, or in DNA hybridization arrays, to identify probes or primers and the like.

In another embodiment of this invention genetically filtered libraries can be used to identify sequence polymorphisms in single copy regions useful as genetic markers in marker assisted breeding programs or in positional cloning strategies.

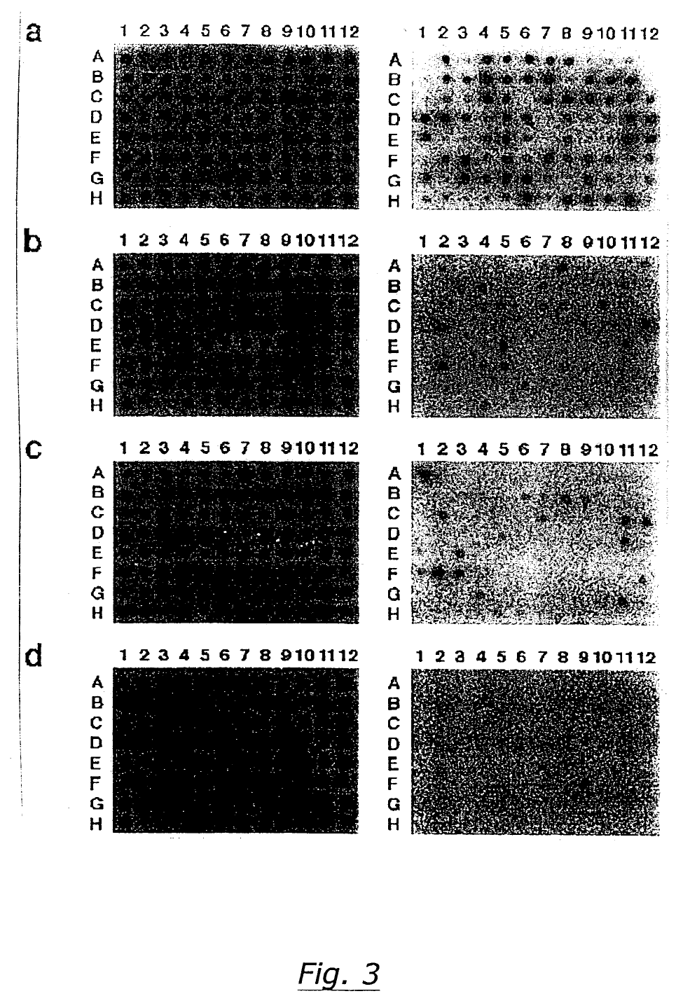

E. coli strains with wild type McrBC and to a lesser extent McrA were previously thought unsuitable for genomic DNA cloning as methylation restriction would prevent the recovery of clones. Grant et. al., P.N.A.S. (1990) Vol 87 P.4645; Woodcock et. al, Nucleic Acids Research (1990) Vol. 25 p.4465; Dogherty et. al, (1991) Gene Vol 98 p. 77; Raleigh et al, Nucleic Acids Research (1988) Vol. 16 p. 1563. These studies, however, were done using bacteriophage lambda vectors in which insert sizes ranged from 15 to 20 kbp (See, e.g., Grant, S. G., et al. (1990) Proc. Natl. Acad. Sci. U.S.A. 87:4645; D. M. Woodcock, et al., (1988) Nucleic Acids Res. 25:4465). The probability of cloning a genome fragment of that size that does not contain repetitive DNA is very low. This problem can be circumvented by the judicious use of small insert libraries. For example, and not limitation, inserts of 0.5 to 4 kbp allowed efficient recovery of maize genes from a filtered library in a comparable proportion to that of much less complex genomes such as rice (See Examples and FIG. 3).

In another embodiment the sequence information generated herein may be compared to the complete and highly accurate sequence of a related genome (e.g. S. cerevisiae, C. elegans, A. thaliana, and rice) to yield all or most of the information desired from the target genome. The information can be used itself to create a database of genetic information that which may be probed. Alternatively, it may be used for selection of primers or for hybridization arrays using solid supports such as glass slides, chips, beads and filters.

The present invention also provides a method for producing a library of diverse polypeptides, further comprising the step of providing proper conditions for vectors to express the DNA fragments.

The use of genetic filtering should allow comprehensive gene discovery via genome sequencing to be considered for extremely large plant genomes such as maize, soybean and wheat. Genetically filtered shotgun sequencing is also applicable to mammalian genomes since repetitive DNA in mammals is densely methylated (Kass, S. U., et al., (1997) Trends Genet. 13:444).

Application of this method will result in considerable savings and will speed up the sequencing of complex eukaryotic genomes by up to ten-fold. For example, and not limitation, a three-fold coverage has been shown to be effective in finding most genes (See, e.g., Bouck, J., et al., (1998) Genome Res. 8:1074). Using a 75% success rate and 500 base read lengths, three-fold coverage of the maize genome would take about 20,000,000 read attempts. A ten-fold increase in efficiency using the genetically filtered shotgun method would give the same approximate data from 2,000,000 reads. Typical cost per read at the time of this application is about $5.00. Hence the application of this invention would save about $90,000,000 in a maize gene discovery program.

General Techniques

The practice of the present invention will employ, unless otherwise indicated, conventional techniques of molecular biology, microbiology, and recombinant DNA technology, that which are within the skill of the art. Such techniques are explained fully in the literature.

In a preferred embodiment the invention comprises construction of genomic libraries in methylation restrictive host strains. For this embodiment the invention comprises host strains with wild-type McrBC and McrA gene products such as found in JM107, JM101 and JM109 ofE. coli, or any other host strain that restricts methylated DNA. The invention can employ any host strain which expresses McrBC and/or McrA gene products, whether transgenic or naturally occurring.

There are a number of ways to introduce genomic DNA into host cells (See, e.g. Watson, J. D., et al. (1992) “Recombinant DNA”, (W. H. Freeman & Co., New York) pp 99-133, incorporated herein by reference). And, all such methods are contemplated here as being useful with the methods of the invention. In one embodiment the invention comprises the use of electroporation. Electroporation is a highly efficient method of introducing DNA into bacteria and other types of cells. (See, e.g. Watson, supra; pp. 221-222).

Partial genomic libraries may be prepared by digesting nuclear genomic DNA with a methylation insensitive enzyme, as for example SpeI. Alternatively, randomly sheared genomic DNA can be used to avoid potential biases imposed from using restriction endonucleases and to facilitate assembly. The two strategies are laid out in Table 1

TABLE I
Genetically Filtered Shotgun
Sequencing

| | |
|---|---|
| Purify nuclear DNA from | Purify nuclear DNA from |
| immature ears | immature ears |
| 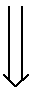 | 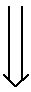 |
| Shear DNA and select 1-4 Kb | Digest with SpeI and select |
| fragments | 1-4 Kb fragments |
| 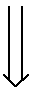 | 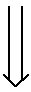 |
| Ligate into M13 | Ligate into XbaI digested |
| | M13 |
| 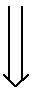 | 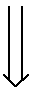 |
| Transform Mcr+E. coli strains | Transform E. coli strains |
| | varying in mcr genotype |
| 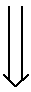 | 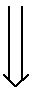 |
| Ed-sequence white plaques | End-sequence 300-400 white |
| | plaques from each |
| 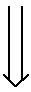 | 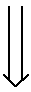 |
| Analyze Sequence | Analyze sequence |

As used herein, a genomic library refers to a mixture of clones constructed by inserting fragments of genomic DNA into a suitable vector. Genomic DNA can be derived from the entire genome, a single chromosome, or a portion of a chromosome. Sources of genomic DNA can be obtained from any nucleated cell, tissue, or organ throughout the life cycle of the organism. It is important to exclude sources of contaminating unmethylated DNA from the genomic DNA to be sequenced. Such sources may include organellar DNA (mitochondrial, or chloroplast (DNA)) from these preparations, however, as this is unmethylated and will also be enriched in the preparation. DNA from microbes and other parasites can also be unmethylated and will also be enriched.

In a preferred embodiment, for maize, nuclear DNA is obtained from a tissue and size fractionated by agarose electrophoresis and spin columns to enrich for 0.5 to 4 kbp fragments if the DNA was restriction enzyme cleaved, or 1.6 to 4 kbp fragments if it was sheared. DNA so prepared is ligated into a cloning vector suitable for propagation in the host strain. Cloning vectors include, but are not limited to those based on the filamentous phage M13. Vectors based on double-stranded plasmids or phage are also appropriate in this context. M13 is a single-stranded, filamentous DNA bacteriophage. The double-stranded replicative form (RF) can be isolated and used as a cloning vector. DNA fragments are ligated into the vector at unique restriction sites, then the recombinant M13 DNA is transformed into E. coli.

M13 cloning vectors were developed to produce single-stranded template DNA for DNA sequence analysis. DNA is ligated into M13 in a region of the vector termed the “polylinker”, so called because it contains many restriction enzyme recognition sequences,that are present only once in the vector. An oligonucleotide primer (i.e. the universal sequencing primer) that anneals adjacent to this polylinker region is used to sequence the inserted DNA fragment. This primer can be used to obtain the DNA sequence from one end of the clone to over 400 bases away (See Watson et al., supra, pp.117-119).

The sequencing step may be carried out either manually or using an automated DNA Sequencer employing methods well known in the art. In a preferred embodiment, one end from each of several clones is subjected to “one pass” (i.e. sequencing only once) automated DNA sequencing as described in the Examples. Automated DNA sequencing devices are well known and widely available to those of skill in the art. For example, and not limitation, sequencing devices are available from Applied Biosystems, Amersham/Pharmacia, and Millopore.

Raw sequence information obtained from automated sequencing can be used any of a number of ways standard in the art. It may be analyzed immediately using on-line parallel processing microcomputers that employ existing software programs adapted for parallel processing. Sequence analysis software programs contemplated for use herein include, for example and not for limitation, BLASTN and BLASTX, which compares sequence similarity between nucleotides and amino acid sequences, respectively (See, e.g., Altschul et al., (1990) J. Mol. Biol. 215:403-410); TBLASTX which programs compare predicted amino acid sequence in all possible reading frames from a simple sequence to the same from a DNA database. More specifically, sequence analysis following the methods of filtering genomic DNA of the present invention can be subjected to matching programs as follows:

Repeat DNA—BLASTN matches to annotated repeats (retroelements, telomeric, centromeric, and knob repeats);

Exon DNA—BLASTX matches E<10-4 against GenBank (mostly rice and Arabidopsis when doing maize comparisons);

Minisatellite DNA—simple sequences without mcrBC sites;

Organellar DNA—BASTN matches to chloroplast or mitochondrial DNA.

All articles cited herein are expressly incorporated in their entirety by reference.

### EXAMPLES

### Example 1

The Maize Genome.

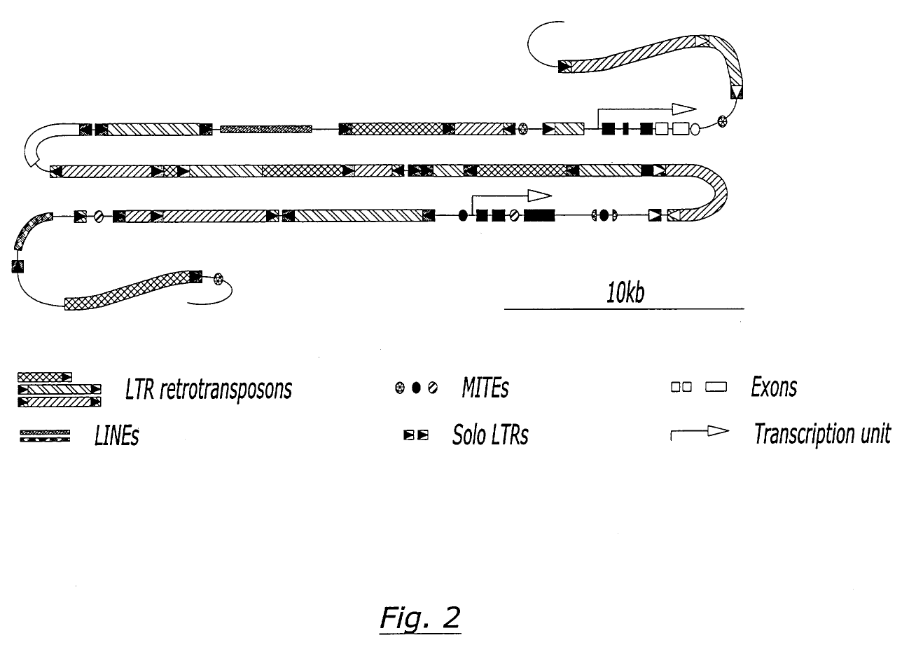

As shown in FIG. 2 (modified from White and Doobley (1998), the maize genome is composed of low copy (gene-rich) regions intermixed with large stretches of repetitive elements which account for 50-80% of the DNA. The haploid genome of maize is estimated to be 2,500 Mb. About 50-80% of the nuclear of maize is composed of nested retrotransposable elements. (See, e.g., SanMiguel, P., et al (1996) Science 274:765; Hake, S. and V. Walbot (1980) Chromosoma 79:251). Introns and untranslated leaders are typically short, but comprise 60% of most genes.

### Example 2

Enrichment for Genes in Filtered Libraries.

The frequency of finding genes (gene density) was estimated in random genomic sequences from maize. A partial genomic library was constructed using maize nuclear DNA from immature ears digested with the methylation insensitive restriction enzyme Spe I and size fractionated to enrich for 0.5 to 4 kbp fragments. Nuclear DNA was isolated by purifying nuclei by standard procedures as follows: 100 g of immature ears from Zea mays inbred B73 were ground in liquid N₂, transferred to a blender with 6 volumes of extraction buffer (25 mM citric acid pH 6.5, 250 mM sucrose and 0.7 Triton X-100) and then homogenized in a Polytron (Sorvall). The homogenate was successively filtered by cheesecloth, 60 micron and 20 micron nylon mesh (Millipore). Nuclei were centrifuged at 800 g for 10 min at 4° C. and washed in 0.1 volume of extraction buffer by centrifuging at 600 g for 10 min at 4° C. and resuspended in 20 ml of Percoll (Sigma) equilibrated with a few drops of 5× extraction buffer. The slurry was centrifuged at 4000 g and the floating nuclei were collected and washed twice as before. The pellet was finally resuspended in urea extraction buffer to purify the DNA by the urea-phenol method (Cone, K.(1989) Maize Genet Coop Newsl 63, 68).

This DNA was ligated into Xba I digested phage M13 vector and introduced into E. coli strain JM107MA2 (See Blumenthal, R. M., et al. (1985.) J. Bacteriol. 164:501). This strain has mutations in the mcrA and mcrBC modification-restriction systems so that methylated DNA is not underrepresented (See Raleigh, E. A. and G. Wilson (1986) Proc. Natl. Acad. Sci. U.S.A. 83:9070).

One end from each clone was sequenced using standard automated procedures as follows: DNA was isolated from M13 clones using the thermal-max procedure (Mardis, 1994). All phage clones were grown and DNA isolated from 96 well plates. Template DNA was then sequenced, also in 96 well plates. The sequencing reactions were carried out using dye primer chemistry (Amersham Energy-transfer primers) and a thermostable polymerase (Thermal Sequenase, Amersham, Inc.). The products of the reactions were analyzed on ABI377 sequencers and Long Ranger gel matrix. Sequence data were transferred from the ABI sequencers following a check on lane tracking and transferred to a Sun workstation for further processing. The bases were called from the raw sequence data using an automated version of the PHRED base calling program. The base calling software automatically removes vector sequence and poor quality sequence at the 3′ end of the sequence reads. Once in the appropriate directory, the sequences were used to search Genbank using BLAST. Software is available that will automatically batch search thousands of sequences in this manner using a single command.

439 clones were end sequenced from the JM107MA2 maize library. For comparison, 340 randomly selected non-overlapping bacterial artificial chromosome (BAC) end sequence reads from rice and 352 from Arabidopsis were downloaded from publicly available internet sites (e.g., http://www/genome.clemson.edu/projects/rice.html; ftp://ftp.tigr.org/pub/data/a_thaliana/). All of these sequences were subjected to sequence similarity searches.

As shown in Table I, 2.3% of the maize sequences (JM107MA2), 13.5% of the rice sequences and 27% of the Arabidopsis sequences showed significant similarity to protein coding sequences in GenBank. The estimated genome size of maize is about 2500 Mbp but as it is a segmental allotetraploid, the haploid maize genome size is 1250 Mbp, about ten times larger than Arabidopsis (See Arumuganathan, K. and E. D. Earle (1991) Plant Mol. Biol. Rep. 9:208; Gaut, B. S., and J. F. Doebley (1997) Proc. Natl. Acad. Sci. U.S.A. 94:6809). In agreement with this estimate, the percentage of genes found in random Arabidopsis BAC ends is about ten times higher than in maize shotgun reads.

Similar maize libraries were constructed in the methylation restrictive E. coli host strains JM101, JM107 and JM109. The three strains were transformed with the same ligation mix used to transform JM107MA2, and several hundred clones were end-sequenced from each library. BLASTN and BLASTX searches were performed against non-redundant nucleotide and protein sequence databases (GenBank-NCBI) and TBLASTX searches were performed against ‘dbEST (GenBank-NCBI) and ‘at_gb’ [Arabidopsis thaliana Genbank sequences collected by AtDb (http://genome.www.stanford.edu/Arabidopsis/dir.html; Flanders, D. J., et al. (1998) Nucleic Acids Res. 26:80)].

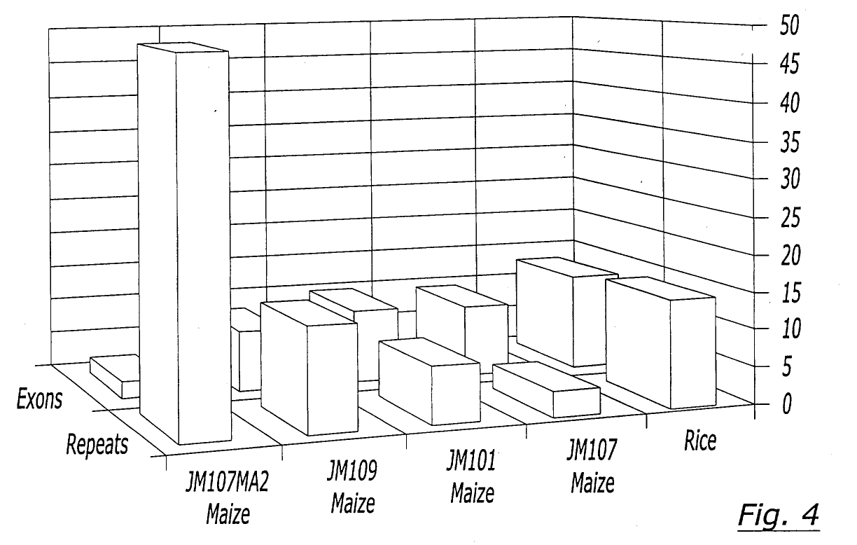

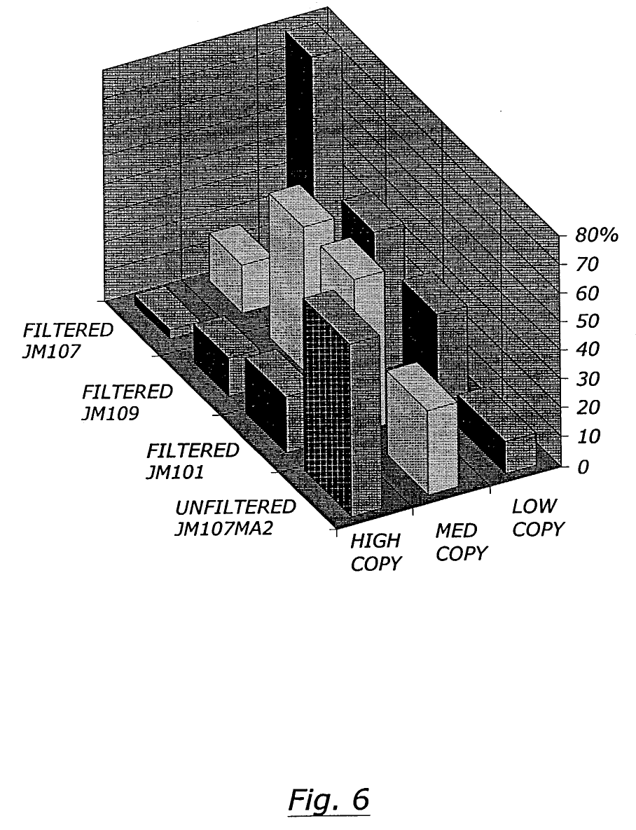

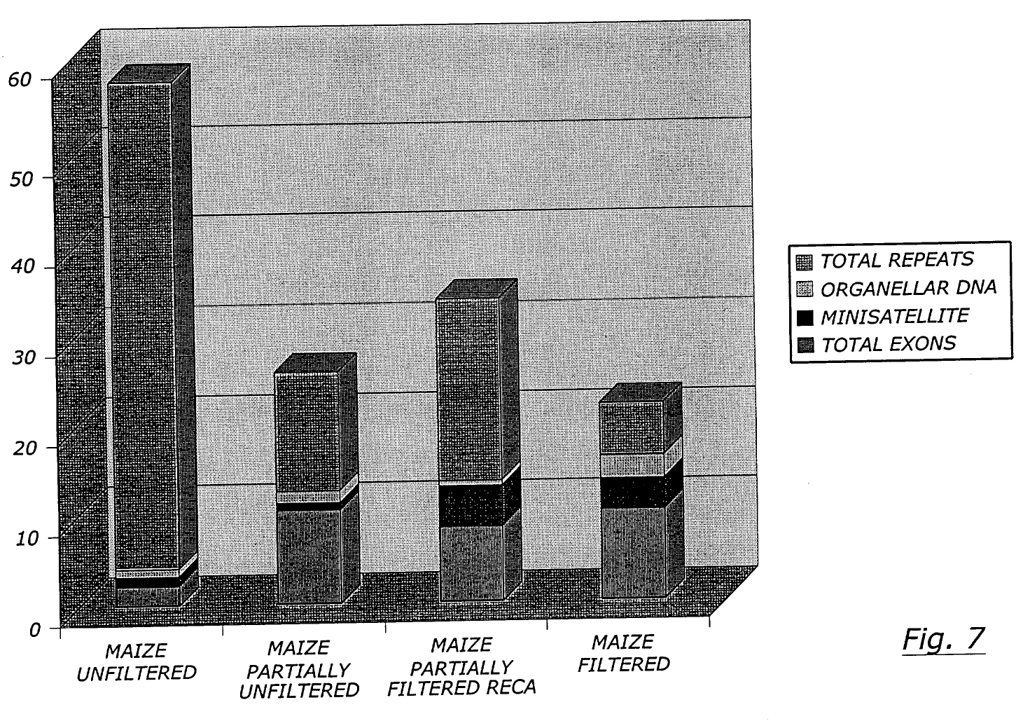

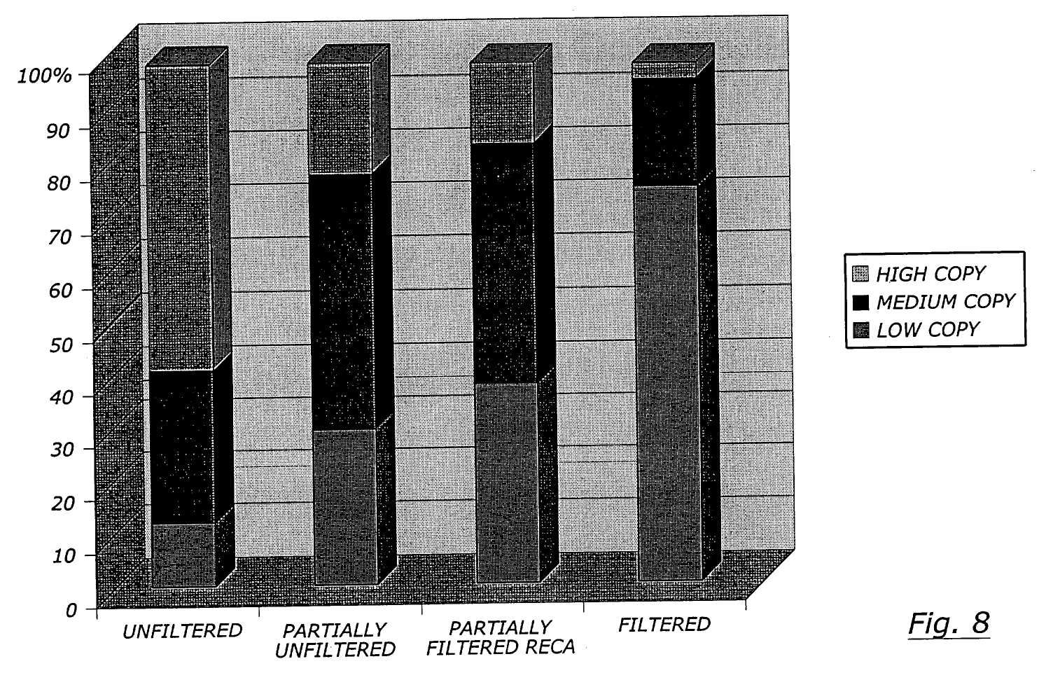

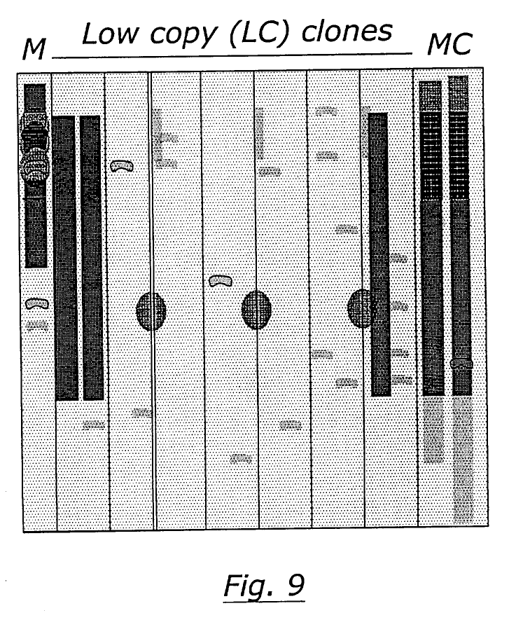

The three genetically filtered libraries had fewer clones containing repetitive DNA than the unfiltered library. For example, 48.7% of the clones propagated in the unfiltered strain matched retro-transposons and other annotated repeats (Table I). In contrast, only 3.3% of the clones propagated in JM107 matched annotated repeats, and less than 10% matched all repetitive sequences. As predicted, the proportion of database matches to known coding sequences was increased four fold in the filtered versus the non-filtered libraries, with some differences between the different strains (Table I). See also FIGS. 4-9. This increased the density of exons detected among maize filtered genomic sequences (i.e. 10%) to nearly that observed in rice (i.e. 13.5%). Given that introns comprise 60% of maize genes, and would not be recognized by protein database searches, it is likely that the actual number of recognizable genes represented in this collection is even higher, approaching 25%. As the number of proteins in public databases increases, the number of recognizable genes will also increase.

An independent estimate of the proportion of clones containing repetitive DNA was obtained by performing dot-blots using 96 clones from each sequencing library. Dot blots were performed using a Hydra-96 pipetting device to spot M13 template DNA onto Hybond nylon membranes. Hybridization was done in Church Buffer (G. M. Church and W. Gilbert (1984) Proc. Natl. Acad. Sci. U.S.A. 81:1991) at 58° C. and washes were done in 0.2×SSC at 58° C. for the genomic DNA probe and at 65° C. for the vector probe. Hybridization probes were labeled by random priming (Boehringer Mannheim) using 10 ng of linearized M13 DNA or approximately 200 ng of nuclear genomic DNA. The four membranes were successively hybridized to total maize nuclear genomic DNA and to an M13 probe for normalization.

In this assay, only clones containing repetitive DNA were expected to display detectable hybridization. High copy sequences are represented in the probe and therefore hybridize at high stringency. Low copy sequences do not hybridize above background. FIG. 2 shows that the best of the filtered libraries, JM107, had the smallest number of hybridizing clones while the unfiltered library, JM107MA2, had a much higher number of hybridizing clones.

Quantitation revealed that 59.1% of the clones in the unfiltered library contained highly repetitive sequences. This compared with only 3.1% of the clones from JM107. Importantly, most of the clones from the unfiltered library whose sequences had no significant match in the database contained high or middle repetitive DNA. In contrast, most of the clones with no significant database match from filtered libraries had low copy DNA.

These results illustrate that use of small insert libraries coupled with restriction of methylated DNA allows maize genes to be recovered efficiently from a filtered library in a comparable proportion to that of much less complex genomes such as rice (see FIG. 3). The enrichment for genes in the filtered libraries was 4-6-fold based on the increase in coding regions or 20-fold based on the reduction of repeats. The proportion of maize genes also may be underestimated because GenBank has many more Arabidopsis and rice genes than maize, thus fewer matches are expected with maize coding regions than with rice or Arabidopsis.

TABLE II

| | Maize | | | | Rice | Arabidopsis |
|---|---|---|---|---|---|---|
| “Haploid” genome size | 1250 | | | | 430 | 120 |
| Library | JM107MA2 | JM101 | JM109 | JM107 | BAC ends | BAC ends |
| E. coil genotype | mcrA− | mcrA+ | mcrA− | mcrA− | mcrA− | mcrA− |
| | mcrBC− | mcrBC+ | mcrBC+ | mcrBC+ | mcrBC− | mcrBC− |
| Number of reads | 439 | 303 | 159 | 242 | 340 | 352 |
| Average read length | 441 bp | 391 bp | 394 bp | 376 bp | 438 bp | 431 bp |
| Annotated repeats* | 48.7% | 7.6% | 13.8% | 3.3% | 14.4% | 7.4% |
| Unannotated repeats^{†} | 5.0% | 5.6% | 6.3% | 2.5% | n.d. | n.d. |
| Minisatellite^{‡} | 0.9% | 0.7% | 4.4% | 3.3% | n.d. | n.d. |
| Known exons^{§} | 1.4% | 8.2% | 6.9% | 8.3% | 10.9% | 20.4% |
| Hypothetical exons^{§} | 0.9% | 2% | 1.3% | 1.6% | 2.6% | 6.5% |
| Total exons^{§} | 2.3% | 10.2% | 8.2% | 9.9% | 13.5% | 27% |
| Organellar DNA^{#} | 0.5% | 1.3% | 0.6% | 2.5% | 2.1% | 0.8% |
| No hybridization (LC)^{¶} | 11.3% | 31.2% | 37.9% | 76.9% | n.d. | n.d. |
| Weak hybridization (MC) | 29.6% | 47.5% | 46.5% | 20% | n.d. | n.d. |
| Strong hybridization ( | 59.1% | 21.2% | 15.5% | 3.1% | n.d. | n.d. |

*transposons, knobs, autonomous replicating sequences, retroviral genes, telomeric and centromeric repeats.
^{†}same GemBank entry hit by different clones, indicating the presence of a repeat.
^{‡}simple sequence repeats detected by BLASTN or BLASTX in various GenBank entries.
^{#}mitochondrial or chloroplast DNA.
*^{†‡#}BLASTN cutoff E < 9.9 10⁻¹², BLASTX or TBLASTX cutoff E < 9.9 10⁻⁵.
^{§}BLASTX cutoff E < 9.9 10⁻⁵.
^{¶}hybridization with radiolabelled total maize DNA (FIG. 2).

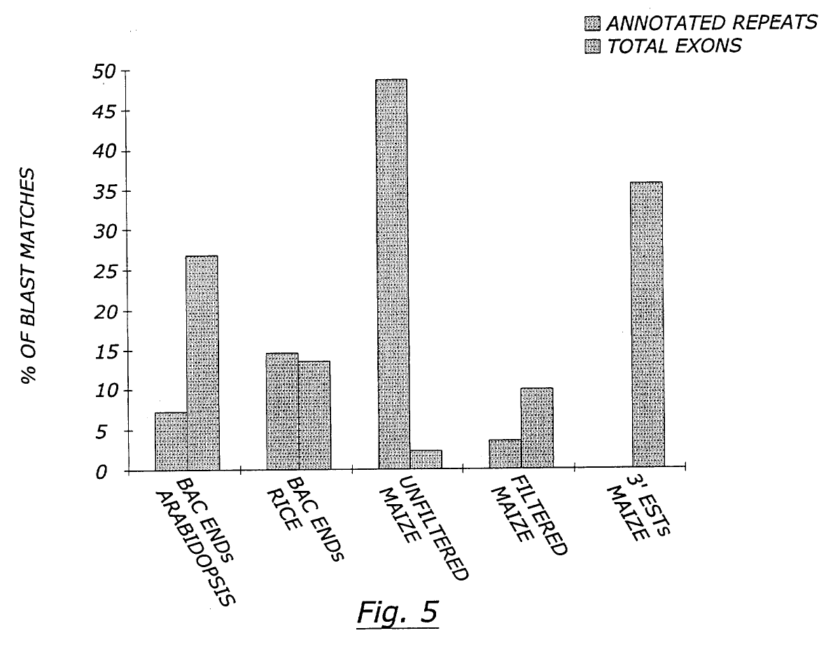

As shown in the table and in FIGS. 5-9, 10% of genetically filtered shotgun reads match exons. The average maize gene is 40% exon, therefore 25% of filtered reads is from known genes. 30-40% of maize ESTs match known exons. Therefore most of the sequence represented in genetically filtered libraries represents genes and intervening sequences. Methylation in the maize genome is primarily restricted to highly repetitive DNA, especially retrotransposons. MCR+ strains can be used to select genes from shotgun libraries. 0.25% of the resulting sequence is from genes, giving a comparable gene density to model genomes such as rice.

### Example 3

(Prophetic)

There are other methods by which repeat and unique DNA containing clones can be separated. At least two methods are possible. We will explore two methods; repeat hybridization in solution and repeat hybridization on filters (‘cold-spot selection’). These are by no means mutually exclusive and in fact might very well be most effective when used in combination.

The small number of repetitive elements provides several avenues for enrichment of clones for unique DNA by the elimination of repetitive DNA.

First one selects a unique DNA by a simple hybridization to remove the high copy DNA. DNA will be isolated from maize, nebulized, and linkers added as before. These fragments will be denatured and then allowed to reanneal so that the high copy number DNA will become double stranded. Double stranded DNA will be removed by hydroxyapatite immobilization, or by restriction enzyme digestion. The single-stranded DNA remaining will be greatly enriched for unique DNA, and will be amplified and cloned into M13.

Alternately one can make a total genomic DNA library in M13 clones. These can be amplified en masse and hybridized back to immobilized genomic DNA in varying ratios. The material not immobilized should be the lower copy number unique DNA.

There has been a technological advance in recent years that enables high density arrays of clones to be plated and hybridized. One can plate grids of randomly cloned maize genomic fragments in M13, using appropriate host strains. The grids are then interrogated with several probes to select those containing repetitive DNA. Clones not hybridizing to these probes (‘cold spots’) will be sequenced.

One probe for testing is total genomic DNA. At the appropriate concentration, which can be empirically determined, the probe will only hybridize strongly to repeat DNA in the subclones due to the relatively higher concentration of this DNA relative to a given region of unique sequence (Shephard et al., 1982; Bennetzen et al., 1994). An example of such a cold-spot hybridization is shown in FIG. 2. Alternately one can test a repeat cocktail, containing DNA from all the known maize repeats. This may be less effective due to the presumably large number of middle repetitive elements in the maize genome which have not all been identified. One should plate about 5000 plaques as a test of this strategy. These are then hybridized with repeat containing probe and the non-hybridizing clones sequenced. Database searches can then be carried out to test the effectiveness of the selection.

### Example 4

(Prophetic)

Removal of Repetitive Sequences by Hybridization

Plant Genomic DNA Isolation

Plant genomic DNA will be isolated using the following protocols from leaf tissues. Briefly, 50 g of frozen tissue will be grounded in liquid Nitrogen using mortar and pestle into fine powder. Tissue powder is transferred immediately into a blender containing ice cold XIB buffer (25 mM of Citric acid, 250 mM of Sucrose, 0.7% of Triton X and 0.1% of BME) at a concentration of 10 ml/g of tissue sample. This solution is homogenized for 10-30 s and is filtered into an ice cold 250 ml centrifuge tube through two layers of cheese cloth and one layer of 60 micrometer nylon mesh (Millipore cat. NY600010). The remaining nuclei will be retrieved by squeezing the homogenates with gloved hands. The homogenate will be centrifuged with a fixed-angle rotor at 2000 g at 40° C. for 15 minutes. The supernatant will be discarded and the pellet will be suspended in 15 ml wash buffer (50 mM of EDTA, 350 mM of Sorbitol, and 0.1% of BME) with wide-bore pipette. 3 ml of 5% Sarcosyl solution will be added into the pellet solution and mixed gently and left at room temperature for 15 minutes. 2.6 ml of 5M NaCl will be added into the solution and mixed gently. 2.1 ml of prewarmed (65° C.) CTAB (8.6% of CTAB, 0.7 M of NaCl) solution will be added and incubated at 65° C. for 15 minutes. The solution then will be phenol-chloroform extracted and isopropanol precipitated. DNA fiber will be washed in 70% ethanol and resuspended in 750 ml of TE buffer.

Fragmentation of Plant Genomic DNA

The above prepared plant genomic DNA will be sheared by mechanical force through the use of a nebulizer. Briefly, 20 μg of genomic DNA will be mixed with 2 ml of nebulization buffer (50 mM of Tris-HCl, pH 8.0, 15 mM of MgCl₂ and 25% of glycerol) on ice. The DNA solution will be nebulized using high purity regulated nitrogen gas for 1 minute at 10 psi, resulting in DNA fragments with a size of 0.5-3.0 kb. DNA will be ethanol precipitated and resuspended in TE.

Plant Genomic DNA Reassociation

DNA samples ranging in concentration from 5 ug/ml to 2.5 mg/ml will be denatured at 100° C. (boiling) for 5 minutes in microcnetrifuge tube and reassociated in 0.12 M sodium phosphate buffer, pH 6.8 at 60° C. The reassociation reactions will be terminated at Cot>100 by quick cooling in an ethanol-dry ice bath. Co is the concentration of single-stranded DNA at the beginning of the reassociation reaction and t is the time of the reassociation reaction. Cot describes the kinetic behavior of DNA reassociation and in plants, majority of the repetitive DNA are reassociated at Cot between 0.01 and 100 (references 1 and 2, immediately below). Fractionation of the single- and double-strand DNA will be achieved by hydroxylapatite (HAP) chromatography. HAP will be preincubated at at 100° C. for 5-10 minutes in 0.12 M sodium phosphate buffer, 0.02% (w/v) sodium lauryl sulfate to reduce non specific DNA retention. Reassociated DNA samples will be thawed and loaded into HAP columns at at 60° C. and single-stranded DNA will be eluted by using 0.12 M sodium phosphate buffer. In HAP column separation of DNA, all DNA is bound at low salt concentrations. At intermediate salt concentrations (0.12 mM), partially or completely double-stranded DNA remains bound to HAP, while single-stranded DNA elutes. The single-stranded DNA elution will then be concentrated and allowed to cool until it also has completed its reannealing.

The following references are specifically incorporated herein to the extent they supplement the methods and materials recited above: 1) Ranjekar, P. K., Pallotta, D., and Lafontaine, J. G (1976). Analysis of the genome of plants Biochem. Biophys. Acta, 425, 30-40; 2) Gurley, W. B., Hepburn, A. G., and Key, J. L (1979). Sequence organization of soybean genome. Biochem. Biophys. Acta, 561, 167-183.

End Repair of DNA Fragments

The ends of the digested DNA will then be repaired using the Klenow fragment of DNA polymerase or T4 polymerase. End repair reaction is performed at RT for 15 min in a total volume of 30 uL containing 22 uL of DNA, 5 mM of MgCl₂, 0.05 mM dNTP mixture, and 10 units of T4 DNA polymerase. After 15 minutes, 5 units of klenow fragment ofE. Coli DNA polymerase I is added and incubated for an additional 15 minutes at RT. The DNA is phenol extracted and then ethanol precipitated. Prior to ligation with the vector, the end fragments are treated with T4 Polynucleotider kinase in a total reaction volume of 30 uL containing 0.05 M Tris-Cl, 10 mM MgCl₂, 5 mM DTT, 2 mM ATP and 1 unit of T4 Polynucleotide kinase. The reaction mixture is incubated at 37° C. for 30 minute and then heat inactivated at 65° C. for 5 minute. The DNA is recovered by ethanol precipitation.

Ligation of Insert and Vector

Ligations are performed in a total reaction of 10 μL. Each ligation contains approximately 50-150 ng of insert DNA and 20 ng of dephosphorylated linearized vector. First the insert DNA and vector plus additional required sterile water are mixed and then ligation buffer and 3 units of T4 DNA ligase (Promega, USA) are added and mixed gently. The ligation mixture is incubated at 16° C. overnight. The ligation reaction is heat inactivated at 65° C. for 10 minutes and stored on ice or at 4° C. In order to avoid arcing during cell transformation by electroporation caused by high salt concentrations in the ligation reaction, the ligation reaction is diluted 5 times.

Transformation and Colony Picking

Twenty μL of E. coli DH5α (Gibco BRL, USA) were transformed with 1 μl of the diluted ligation reaction mixture using a BioRad gene pulser with the following settings: 1.8 KV, 200 ohm low range resistance, and 25 V capacitance. Transformed cells are transferred into 0.5 mL of SOC (2% Bactotryptone; 0.5% Bacto yeast extract; 2.5 mM KCl; 10 mM NaCl; 10 mM MgCl₂; 10 mM MgSO₄; 20 mM glucose, pH 7.0) and shaken in a 37° C. incubator at 225 rpm for one hour. Cells are spread onto LB media containing 30 μg chloramphenicol, X-Gal, and IPTG and grown at 37° C. for 18 hours. The plates are then transferred to the dark and stored at room temperature for an additional 1-2 days to allow the nonrecombinant colonies to turn dark blue making colony picking more efficient. Recombinant clones are picked and stored in 384 well microtiter master plates containing LB freezing media (36 mM K₂HPO₄; 13.2 mM KH₂PO₄; 1.7 mM sodium citrate; 0.4 mM MgSO₄, 6.8 mM (NH₄)₂SO₄; 4.4% glycerol (v/v); 12.5 μg/mL chloramphenicol). Prior to any further library use, a replica of the library is produced (working copy 1) from the master copy and stored in separate −80° C. freezers.

### Example 5

(Prophetic)

In Vitro Restriction of Methylated DNA

As an alternative to using strains of bacteria that inherently express methylation dependent restriction enzymes, it is possible to use purified enzymes to digest genomic DNA into small fragments before size fractionation and cloning.

For example, the enzyme encoded by mcrBC from New England Biolabs (Beverly, Mass.) digests methylated DNA into fragments of 80-100 bp on average. Gel fractionation of digested DNA can then be used to isolate larger fragments of unmethylated DNA.

There are several technical hurdles that must be addressed with this approach. First, overloading of the gel may occur as most DNA will be in the 100 bp range. This will cause smearing and poor fractionation. However, this should be overcome by running the electrophoreses gel at low voltage such as 1V/cm and longer time. Second, digestion must be partial: if every available site were digested it would be impossible to recover overlapping clones for sequence assembly. (presumably the unmethylated fragments would be the genes so each fragment could be sequenced completely to obtain gene coverage ie. It is not necessary to have overlapping clones and in fact it may be more desirable to have full length clone. A disadvantage is that you will have fewer entry points into each gene from an end sequencing strategy.

Plant DNA will be digested with mcrBC using a series of time points. The time points are selected in order that the DNA is digested to a few hundred bp on average. The DNA will be next be selectively separated using gel electrophoresis to recover fragments in the 1-4 kb range. The fractionation will be repeated in order to remove small fragments inadvertently recovered by overloading. The ends of the digested DNA so recovered will then be repaired using the Klenow fragment of DNA polymerase or T4 polymerase or similar standard technique such as Mungbean nuclease treatment. Finally, clones will be created in order to isolate fractions into plasmid or phage vector for sequencing using DNA linkers and other standard techniques.

General Method #1

Genomic DNA Isolation

Plant genomic DNA will be isolated using the following protocols from leaf tissues. Briefly, 50 g of frozen tissue will be grounded in liquid Nitrogen using mortar and pestle into fine powder. Tissue powder is transferred immediately into a blender containing ice cold XIB buffer (25 mM of Citric acid, 250 mM of Sucrose, 0.7% of Triton X and 0.1% of BME) at a concentration of 10 ml/g of tissue sample. This solution is homogenized for 10-30 s and is filtered into an ice cold 250 ml centrifuge tube through two layers of cheese cloth and one layer of 60 micrometer nylon mesh (Millipore cat. NY600010). The remaining nuclei will be retrieved by squeezing the homogenates with gloved hands. The homogenate will be centrifuged with a fixed-angle rotor at 2000 g at 40° C. for 15 minutes. The supernatant will be discarded and the pellet will be suspended in 15 ml wash buffer (50 mM of EDTA, 350 mM of Sorbitol, and 0.1% of BME) with wide-bore pipette. 3 ml of 5% Sarcosyl solution will be added into the pellet solution and mixed gently and left at room temperature for 15 minutes. 2.6 ml of 5M NaCl will be added into the solution and mixed gently. 2.1 ml of prewarmed (65° C.) CTAB (8.6% of CTAB, 0.7 M of NaCl) solution will be added and incubated at 65° C. for 15 minutes. The solution then will be phenol-chloroform extracted and isopropanol precipitated. DNA fiber will be washed in 70% ethanol and resuspended in 750 ml of TE buffer.

DNA Fragmentation and Size Selection

The above prepared plant genomic DNA will be sheared by mechanical force through the use of a nebulizer. Briefly, 20 μg of genomic DNA will be mixed with 2 ml of nebulization buffer on ice. The DNA solution will be nebulized using high purity regulated nitrogen gas for 1 minute at 10 psi, resulting in DNA fragments with a size of 0.5-4.0 kb. DNA will be ethanol precipitated and resuspended in TE. DNA fragments are separated by electrophoresis on a 1% agarose gel in 1×TAE with a constant voltage of 1.0 V/cm for 12 hours. After staining with 0.5 μg/μL of Ethidium bromide, the fragments ranging from 1-4 kb are selected.

Extraction of Size-Selected DNA Fragments

DNA fragments in gel slices are recovered by common method such as electroelution or phenol extraction. For electroelution, approximately 100 mg of DNA slices of each fraction are equilibrated in 1×TAE on ice for 1 hour. The equilibrated DNA slice is then placed in the electroelution chamber with 200 μL 1×TAE and the electroelution is performed at 4° C., 4.5 V/cm for 1 hours in 1×TAE in an electrophoresis chamber. The electroelution chamber used has a capacity to retain small molecules (Spectraphor, USA). To release any membrane-bound DNA molecules, the polarity is switched for 2 min and the electroeluted DNA is recovered. The DNA can the be concentrated using ethanol precipitation and resuspended in 22 μL of TE. DNA ranging from 1-4 kb are combined.

McrBC Digestion and Second Size Selection

Size-selected DNA is digested completely with McrBC in a total volume of 30 μL at 37° C. water bath for 1 hour and inactivated by adding 1/10 volume of 0.5 M EDTA (pH 8.0). The non digested DNA can then be separated from digested methylated DNA by electrophoresis on a 1% agarose gel in 1×TAE with a constant voltage of 0.5 V/cm for 12 hour. the gel was stained with ethidium bromide at 0.5 μg/mL. DNA fragments ranging from 1-4 kb are again gel-extracted by the same method mentioned earlier and resuspended in 22 μL TE.

End Repair of DNA Fragments

The ends of the digested DNA will then be repaired using the Klenow fragment of DNA polymerase or T4 polymerase. End repair reaction is performed at RT for 15 min in a total volume of 30 μL containing 22 μL of DNA, 5 mM of MgCl₂, 0.05 mM DNTP mixture, and 10 units of T4 DNA polymerase. After 15 minutes, 5 units of klenow fragment of E. Coli DNA polymerase I is added and incubated for an additional 15 minutes at RT. The DNA is phenol extracted and then ethanol precipitated. Prior to ligation with the vector, the end fragments are treated with T4 Polynucleotider kinase in a total reaction volume of 30 uL containing 0.05 M Tris-Cl, 10 mM MgCl₂, 5 mM DTT, 2 mM ATP and 1 unit of T4 Polynucleotide kinase. The reaction mixture is incubated at 37° C. for 30 minute and then heat inactivated at 65° C. for 5 minute. The DNA is recovered by ethanol precipitation.

Ligation of Insert and Vector

Ligations are performed in a total reaction of 10 μL. Each ligation contains approximately 50-150 ng of insert DNA and 20 ng of dephosphorylated linearized vector. First the insert DNA and vector plus additional required sterile water are mixed and then ligation buffer and 3 units of T4 DNA ligase (Promega, USA) are added and mixed gently. The ligation mixture is incubated at 16° C. overnight. The ligation reaction is heat inactivated at 65° C. for 10 minutes and stored on ice or at 4° C. In order to avoid arcing during cell transformation by electroporation caused by high salt concentrations in the ligation reaction, the ligation reaction is diluted 5 times.

Transformation and Colony Picking

Twenty μL of E. coli DH5α (Gibco BRL, USA) were transformed with 1 μl of the diluted ligation reaction mixture using a BioRad gene pulser with the following settings: 1.8 KV, 200 ohm low range resistance, and 25 V capacitance. Transformed cells are transferred into 0.5 mL of SOC (2% Bactotryptone; 0.5% Bacto yeast extract; 2.5 mM KCl; 10 mM NaCl; 10 mM MgCl₂; 10 mM MgSO₄; 20 mM glucose, pH 7.0) and shaken in a 37° C. incubator at 225 rpm for one hour. Cells are spread onto LB media containing 30 μg chloramphenicol, X-Gal, and IPTG and grown at 37° C. for 18 hours. The plates are then transferred to the dark and stored at room temperature for an additional 1-2 days to allow the nonrecombinant colonies to turn dark blue making colony picking more efficient. Recombinant clones are picked and stored in 384 well microtiter master plates containing LB freezing media (36 mM K₂HPO₄; 13.2 mM KH₂PO₄; 1.7 mM sodium citrate; 0.4 mM MgSO₄, 6.8 mM (NH₄)₂SO₄; 4.4% glycerol (v/v); 12.5 μg/mL chloramphenicol). Prior to any further library use, a replica of the library is produced (working copy 1) from the master copy and stored in separate −80° C. freezers.

General Method #2

Genomic DNA Isolation

Plant genomic DNA will be isolated using the following protocols from leaf tissues. Briefly, 50 g of frozen tissue will be grounded in liquid Nitrogen using mortar and pestle into fine powder. Tissue powder is transferred immediately into a blender containing ice cold XIB buffer (25 mM of Citric acid, 250 mM of Sucrose, 0.7% of Triton X and 0.1% of BME) at a concentration of 10 ml/g of tissue sample. This solution is homogenized for 10-30 s and is filtered into an ice cold 250 ml centrifuge tube through two layers of cheese cloth and one layer of 60 micrometer nylon mesh (Millipore cat. NY600010). The remaining nuclei will be retrieved by squeezing the homogenates with gloved hands. The homogenate will be centrifuged with a fixed-angle rotor at 2000 g at 40° C. for 15 minutes . The supernatant will be discarded and the pellet will be suspended in 15 ml wash buffer (50 mM of EDTA, 350 mM of Sorbitol, and 0.1% of BME) with wide-bore pipette. 3 ml of 5% Sarcosyl solution will be added into the pellet solution and mixed gently and left at room temperature for 15 minutes. 2.6 ml of 5M NaCl will be added into the solution and mixed gently. 2.1 ml of prewarmed (65° C.) CTAB (8.6% of CTAB, 0.7 M of NaCl) solution will be added and incubated at 65° C. for 15 minutes. The solution then will be phenol-chloroform extracted and isopropanol precipitated. DNA fiber will be washed in 70% ethanol and resuspended in 750 ml of TE buffer.

DNA Fragmentation

The above prepared plant genomic DNA will be sheared by mechanical force through the use of a nebulizer. Briefly, 20 μg of genomic DNA will be mixed with 2 ml of nebulization buffer on ice. The DNA solution will be nebulized using high purity regulated nitrogen gas for 1 minute at 10 psi, resulting in DNA fragments with a size of 0.5-4.0 kb. DNA will be ethanol precipitated and resuspended in 80 uL of TE.

End Repair of Fragmented DNA and Size Selection

The ends of sheared DNA is repaired using Mungbean nuclease to provide blunt-ended fragments. The reaction is performed in a total volume of 100 μL containing 80 units of Mungbean Nuclease at 37° C. for 13 minutes. The Mungbean nuclease was inactivated and removed by using phenol-chloroform extraction and ethanol precipitation. DNA fragments are separated by electrophoresis on a 1% agarose gel in 1×TAE with a constant voltage of 1.0 V/cm for 12 hours. After staining with 0.5 μg/μL of Ethidium bromide, the fragments ranging from 1-4 kb are selected.

Extraction of Size-Selected DNA Fragments

DNA fragments in gel slices are recovered by common method such as electroelution or phenol extraction. For electroelution, approximately 100 mg of DNA slices of each fraction are equilibrated in 1×TAE on ice for 1 hour. The equilibrated DNA slice is then placed in the electroelution chamber with 200 μL 1×TAE and the electroelution is performed at 4° C., 4.5 V/cm for 1 hours in 1×TAE in an electrophoresis chamber. The electroelution chamber used has a capacity to retain small molecules (Spectraphor, USA). To release any membrane-bound DNA molecules, the polarity is switched for 2 min and the electroeluted DNA is recovered. The DNA can the be concentrated using ethanol precipitation and resuspended in TE. DNA ranging from 1-4 kb are combined.

Ligation of Insert and Vector

Ligations are performed in a total reaction of 30 μL. Each ligation contains approximately 500-1000 ng of insert DNA and 50 ng of dephosphorylated linearized vector. First the insert DNA and vector plus additional required sterile water are mixed and then ligation buffer and 15 units of T4 DNA ligase (Promega, USA) are added and mixed gently. The ligation mixture is incubated at 16° C. overnight. The ligation reaction is heat inactivated at 65° C. for 10 minutes and stored on ice or at 4° C.

McrBC Digestion of Ligation Reaction

The ligation reaction in the amount of 30 μL can be used directly for McrBC digestion in a total volume of 100 μL (20 units of McrBC, 50 mM NaCl, 10 mM Tris-Cl, 10 mM MgCl₂, 1 mM DTT and 1 mM GTP) at 37° C. water bath for 1 hour. The digestion reaction is phenol-extracted and then ethanol precipitated using yeast tRNA (Gibco BRL, USA) as a carrier and resuspended in 10 μl H₂O

Transformation and Colony Picking

Twenty μL of E. coli DH5α (Gibco BRL, USA) were transformed with 1 μl of the ligation reaction mixture using a BioRad gene pulser with the following settings: 1.8 KV, 200 ohm low range resistance, and 25 V capacitance. Transformed cells are transferred into 0.5 mL of SOC (2% Bactotryptone; 0.5% Bacto yeast extract; 2.5 mM KCl; 10 mM NaCl; 10 mM MgCl₂; 10 mM MgSO₄; 20 mM glucose, pH 7.0) and shaken in a 37° C. incubator at 225 rpm for one hour. Cells are spread onto LB media containing 30 μg chloramphenicol, X-Gal, and IPTG and grown at 37° C. for 18 hours. The plates are then transferred to the dark and stored at room temperature for an additional 1-2 days to allow the nonrecombinant colonies to turn dark blue making colony picking more efficient. Recombinant clones are picked and stored in 384 well microtiter master plates containing LB freezing media (36 mM K₂HPO₄; 13.2 mM KH₂PO₄; 1.7 mM sodium citrate; 0.4 mM MgSO₄, 6.8 mM (NH₄)₂SO₄; 4.4% glycerol (v/v); 12.5 μg/mL chloramphenicol). Prior to any further library use, a replica of the library is produced (working copy 1) from the master copy and stored in separate −80° C. freezers.

### Example 6

(Prophetic)

Methyl-Binding Protein

Plant Genomic DNA Preparation

Plant genomic DNA will be isolated and fragmented as described infra. DNA fractions of size 1-4 kb will be recovered after gel electrophoresis. DNA will be purified from gel slices and phenol-chloroform extracted and ethanol precipitated. DNA is then resuspended in TE buffer.

Preparation of Methyl Binding Protein Column

The MBD column was prepared and tested essentially as described in the reference cited immediately below. Briefly, 30 mg of histidine-tagged methyl-CpG binding domain protein, purified from crude bacterial extracts, was coupled to 1 ml of Ni2+-NTA-agarose (Qiagen) by mixing the protein with the matrix and washing with 8 mM imidazole, 50 mM NaCl, 20 mM HEPES pH 7.9, 10% glycerol, 0.1% Triton X-100, 10 mM β-Mecaptoethanol and 0.5 mM PMSF. The protein is then packed into a HR 5/5 column (Pharmacia).

Separation of Genomic DNA on Me-Binding Protein Column

Heavily methylated DNA fragments elute between 0.6 and 0.85 M NaCl depending on the amount of methyl binding protein on the column. DNAs were loaded onto, washed and eluted from the column in 20 mM HEPES (pH 7.9), 10% glycerol, 0.1% Triton X-100, 0.5 mM PMSF and a NaCl concentration of 0.4 based on previous observation that elution of the DNA with 0.4 M NaCl yield mostly unmethylated DNA fractions (1). Eluted unmethylated DNA is ethanol precipitated and resuspended in TE.

End Repair of DNA Fragments

The ends of the digested DNA will then be repaired using the Klenow fragment of DNA polymerase or T4 polymerase. End repair reaction is performed at RT for 15 min in a total volume of 30 μL containing 22 μL of DNA, 5 mM of MgCl₂, 0.05 mM DNTP mixture, and 10 units of T4 DNA polymerase. After 15 minutes, 5 units of klenow fragment of E. Coli DNA polymerase I is added and incubated for an additional 15 minutes at RT. The DNA is phenol extracted and then ethanol precipitated. Prior to ligation with the vector, the end fragments are treated with T4 Polynucleotider kinase in a total reaction volume of 30 uL containing 0.05 M Tris-Cl, 10 mM MgCl₂, 5 mM DTT, 2 mM ATP and 1 unit of T4 Polynucleotide kinase. The reaction mixture is incubated at 37° C. for 30 minute and then heat inactivated at 65° C. for 5 minute. The DNA is recovered by ethanol precipitation.

Ligation of Insert and Vector

Ligations are performed in a total reaction of 10 μL. Each ligation contains approximately 50-150 ng of insert DNA and 20 ng of dephosphorylated linearized vector. First the insert DNA and vector plus additional required sterile water are mixed and then ligation buffer and 3 units of T4 DNA ligase (Promega, USA) are added and mixed gently. The ligation mixture is incubated at 16° C. overnight. The ligation reaction is heat inactivated at 65° C. for 10 minutes and stored on ice or at 4° C. In order to avoid arcing during cell transformation by electroporation caused by high salt concentrations in the ligation reaction, the ligation reaction is diluted 5 times.

Transformation and Colony Picking

Twenty μL of E. coli DH5α (Gibco BRL, USA) were transformed with 1 μl of the diluted ligation reaction mixture using a BioRad gene pulser with the following settings: 1.8 KV, 200 ohm low range resistance, and 25 V capacitance. Transformed cells are transferred into 0.5 mL of SOC (2% Bactotryptone; 0.5% Bacto yeast extract; 2.5 mM KCl; 10 mM NaCl; 10 mM MgCl₂; 10 mM MgSO₄; 20 mM glucose, pH 7.0) and shaken in a 37° C. incubator at 225 rpm for one hour. Cells are spread onto LB media containing 30 μg chloramphenicol, X-Gal, and IPTG and grown at 37° C. for 18 hours. The plates are then transferred to the dark and stored at room temperature for an additional 1-2 days to allow the nonrecombinant colonies to turn dark blue making colony picking more efficient. Recombinant clones are picked and stored in 384 well microtiter master plates containing LB freezing media (36 mM K₂HPO₄; 13.2 mM KH₂PO₄; 1.7 mM sodium citrate; 0.4 mM MgSO₄, 6.8 mM (NH₄)₂SO₄; 4.4% glycerol (v/v); 12.5 μg/mL chloramphenicol). Prior to any further library use, a replica of the library is produced (working copy 1) from the master copy and stored in separate −80° C. freezers.

The following reference is specifically incorporated herein to the extent it supplements the methods and materials recited above: Cross, S. H., Charlton, J. A., Nan, X. and Bird, A. P. (1994) Purification of CpG islands using a methylated DNA binding column. Nature Genet., 6, 236-244.

What is claimed is:

1. A genomic cloning method for identifying DNA segments containing genes in complex genomes, said method comprising:
constructing a genomic library in an environment that eliminates repetitive sequences, said library comprising fragments of genomic DNA;
inserting said genomic DNA into a suitable vector, and characterizing said DNA segment.

2. The method of claim 1 further comprising the step of randomly shearing said genomic DNA for insertion into said vector.

3. The method of claim 1 further comprising the steps of size fractionating said genomic DNA.

4. The method of claim 1 wherein the restrictive environment is established using mechanical size fractionation of said DNA and selective hybridization followed by hydroxylapatite chromatography.

5. The method of claim 1 wherein said hydroxylapitite selective hybridization is conducted at Cot values between 0.01 and 100.

6. The method of claim 1 wherein the size fractionated DNA fragments are fragments of a size smaller than the size of uninterrupted genetic sequences in the genomic DNA.

7. The method of claim 1 wherein the size fractionated DNA fragments range from about 0.5 to about 4 kilobase pairs.

8. The method of claim 1 wherein said vector is selected from a group consisting of: phage, plasmid or other suitable vectors.

9. The method of claim 1 wherein said phage vector is M13.

10. The method of claim 1 wherein said complex genome is a plant genome.

11. The method of claim 1 where said genome is a cereal grain genome.

12. The method of claim 8 wherein said plant genome is selected from the group consisting of: maize, rice, Brassica, soybean, and wheat.

13. The method of claim 1 wherein said complex genome is a mammalian genome.

14. A genomic cloning method for identifying DNA segments containing genes in complex genomes, said method comprising:
constructing a genomic library in an environment that eliminates repetitive sequences, said library comprising fragments of genomic DNA;
said environment established using mechanical shearing of said genomic DNA, selective hybridization at Cot values of between approximately 0.01 and 100, followed by hydroxylapatite chromatography;
inserting said genomic DNA into a suitable vector, and characterizing said DNA segment.

15. A genomic cloning method for identifying DNA segments containing genes in complex genomes, said method comprising:
constructing a genomic library in a methylation restrictive environment, said library comprising fragments of genomic DNA;
inserting said genomic DNA into a suitable vector, and characterizing said DNA segment.

16. The method of claim 15 further comprising the step of randomly shearing said genomic DNA for;insertion into said vector.

17. The method of claim 15 further comprising the steps of size fractionating said genomic DNA.

18. The method of claim 15 wherein the methylation restrictive environment comprises cell extracts of methylation restrictive bacteria: mcrA⁺/mcrBC⁺, mcrA⁻/mcrBC⁺ or mcrA⁺/mcrBC⁻, or any other methylation restriction system that has similar properties to the mcr system.

19. The method of claim 18 wherein said methylation restrictive bacterial strain is selected from a group comprising: JM101, JM107, and JM109.

20. The method of claim 15 wherein the methylation restrictive environment comprises cell-free enzyme encoded by a methylation restrictive bacterial gene: mcrA⁺/mcrBC⁺, mcrA⁻/mcrBC⁺ or mcrA⁺/mcrBC⁻, or any other methylation restriction system that has similar properties to the mcr system.

21. The method of claim 15 wherein the methylation restrictive environment comprises cell-free enzyme encoded by mcrBC.

22. The method of claim 15 wherein the size fractionated DNA fragments are fragments of a size smaller than the size of uninterrupted genetic sequences in the genomic DNA.

23. The method of claim 15 wherein the size fractionated DNA fragments range from about 0.5 to about 4 kilobase pairs and the DNA is cleaved with a methylation insensitive restriction enzyme.

24. The method of claim 15 wherein a methylation insensitive endonuclease is employed to generate DNA fragments.

25. The method of claim 23 wherein said methylation insensitive endonuclease is Spe I.

26. The method of claim 15 wherein said vector is selected from a group consisting of: phage, plasmid or other suitable vectors.

27. The method of claim 15 wherein said phage vector is M13.

28. The method of claim 15 wherein said complex genome is a plant genome.

29. The method of claim 15 where said genome is a cereal grain genome.

30. The method of claim 28 wherein said plant genome is selected from the group consisting of: maize, rice, Brassica, soybean, and wheat.

31. The method of claim 15 wherein said complex genome is a mammalian genome.

32. A genomic cloning method for identifying DNA segments containing genes in complex genomes, said method comprising:
constructing a genomic library in an environment that eliminates repetitive sequences by selective protein binding of said genomic DNA, said library comprising fragments of genomic DNA;
inserting said genomic DNA into a suitable vector, and characterizing said DNA segment.

33. The method of claim 32 further comprising the step of randomly shearing said genomic DNA for insertion into said vector.

34. The method of claim 32 further comprising the steps of size fractionating said genomic DNA.

35. The method of claim 32 wherein the restrictive environment is established using mechanical size fractionation of said DNA and by methyl-CpG binding domain protein chromatography.

36. The method of claim 32 wherein the size fractionated DNA fragments are fragments of a size smaller than the size of uninterrupted genetic sequences in the genomic DNA.

37. The method of claim 32 wherein the size fractionated DNA fragments range from about 0.5 to about 4 kilobase pairs.

38. The method of claim 32 wherein said vector is selected from a group consisting of: phage, plasmid or other suitable vectors.

39. The method of claim 32 wherein said phage vector is M13.

40. The method of claim 32 wherein said complex genome is a plant genome.

41. The method of claim 32 where said genome is a cereal grain genome.

42. The method of claim 40 wherein said plant genome is selected from the group consisting of: maize, rice, Brassica, soybean, and wheat.

43. The method of claim 40 wherein said complex genome is a mammalian genome.

44. The method of claim 32 wherein said column chromatography is conducted using an elution buffer of approximately 0.4 M NaCl.

45. The method of claim 32 wherein said column chromatography is conducted using Ni2+ NTA agarose.
